(19) United States
(12) Patent Application Publication
(10) Pub. No.: US 2012/0157762 A1
(43) Pub. Date: Jun. 21, 2012

(54) SYSTEM AND METHOD FOR TREATING ERECTILE DYSFUNCTION

(76) Inventor: Ryan Darnell, Eden Prairie, MN (US)

(21) Appl. No.: 13/326,958

(22) Filed: Dec. 15, 2011

Related U.S. Application Data
(60) Provisional application No. 61/423,795, filed on Dec. 16, 2010.

(51) Int. Cl. A61F 5/41

(52) U.S. Cl. 600/40

(57) ABSTRACT

A system and related method for treating ED in a patient is provided. The system can include a small cuff or other selective restriction mechanism or device implanted at or along the existing veins of the penis to control blood flow out of the penis. As such, the system can utilize blood pressure from the heart to achieve and maintain an erection by restricting blood flow out of the penis.

### PRIORITY CLAIM AND RELATED APPLICATION

The present application claims the benefit of U.S. Provisional Application Ser. No. 61/423,795, filed Dec. 16, 2010, and entitled “SYSTEM AND METHOD FOR TREATING ERECTILE DYSFUNCTION” which is incorporated herein in its entirety by reference.

### FIELD OF THE INVENTION

The present invention is generally related to implantable systems for the treatment of erectile dysfunction in men. More specifically, the present invention is directed to an implantable system that uses the existing circulatory system to create and maintain an erection in a patient.

### BACKGROUND OF THE INVENTION

Erectile dysfunction (“ED”) is believed to affect more than ninety million men in the United States and Europe, with seventeen million presenting with severe conditions that greatly interfere with the ability to initiate and maintain erections. ED may arise from a number of causes. Age brings on a lack of arterial elasticity in vessels supplying blood to erectile tissues. Damage to nerves necessary for initiating and sustaining erections brought on by chronic conditions (such as diabetes) or by injury can lead to dysfunction. A significant cause of nerve damage comes from injury that occurs during prostate surgeries, especially radical prostatectomies. Although new surgical procedures have been introduced that conserve the nerves in this region, a majority of men who undergo such procedures can still expect some degree of post-operative ED.

A number of oral medications for treating ED have entered the marketplace in recent years, including VIAGRA, CIALIS and LEVITRA. These medications all provide significant relief to a large segment of men with ED. However, they each require that the medication be taken in advance of initiation of sexual activity and their effects may be delayed if ingested with food. Furthermore, the effectiveness of such drugs can vary greatly from patient to patient, and is even ineffective in a large cross-section of patients.

Various treatments have also been tried in connection with ED, including a muscle constrictors or relaxers, such as Prostaglandin E1, administered by injection into the corpus cavernosum of the penis, positioning a suppository within the urethra and/or topically. These approaches allow for less advance preparation, but are neither consistently effective nor desirable applications across patient populations, especially radical prostatectomy patients.

Implantable devices are also often used to address ED, especially when medications are ineffective or contraindicated. Typically, the implantable devices, such as implantable penile prosthetics, comprise at least one collapsible support that deploys to create an erection and retracts to conceal the support(s). However, these devices are often fully implanted within the patient's body, typically with a substantial portion of the device being implanted within the corpus cavernosum. As a result, the surgical procedure is often highly invasive often requiring positioning of multiple components throughout the pelvic region and reproductive organ. In addition, the implantation procedure can require a large incision and/or placement of temporary tubing to maintain the urology and reproductive functions of the penis following implantation and healing. Patients can become discouraged from pursuing this form of treatment by the extensive nature of the surgery. In addition, penile implants are an end stage treatment, and it is often desirable to provide treatment earlier in the disease state. Thus, there is a desire to obtain a minimally invasive yet effective and durable solution to treat ED that can be used with minimal to no side effects.

### SUMMARY OF THE INVENTION

The present invention is directed to treating ED by placing a constrictive cuff around at least one of the veins leaving one or both of corpus cavernosum of the penis. The constrictive cuff can be selectively tightened around the vein to reduce the inner diameter of the vein to create a back pressure within the corpus cavernosum to maintain the erection. The implantation of the cuff is minimally invasive and maintains the natural circulatory system of reproductive system. Unlike implantable devices where a support is positioned within the corpus cavernosum, the cuff assists in the normal operation of the penis without disrupting the urological or reproductive function of the penis. Similarly, in one aspect, the cuff is sized to maintain the flow of blood through the vein while creating the back pressure within the corpus cavernosum. The flow through the vein is maintained to prevent over pressuring of the corpus cavernosum.

An implantable system for treating ED, according to an aspect of the present invention, comprises a constrictive cuff and an inflation system for controlling the tightening of the constrictive cuff. The constrictive cuff can further comprise a supportive backing and at least one inflatable chamber. In one aspect, the supportive backing can be positioned around the vein such that the inflatable chambers are positioned against the vein. The supportive back can be inelastic such that inflating the chambers tightens the chambers against the vein and reduces the effective inner diameter of the vein to create back pressure within the corpus cavernosum. Similarly, deflating the chambers allows the vein to re-expand and allow normal flow of the blood through the vein. In one aspect, the inflatable chamber or chambers can be sized and spaced along the backing such that inflation of the chambers presses the chambers against all sides of the vein to prevent crimping of the vein wall. In one aspect, the chamber or chambers can be shaped to bend the walls of the vein such that the inner diameter of the vein narrows to at least one choke point when the chambers are inflated. Alternatively, the chambers can be shaped to compress the walls evenly to maintain an constant inner diameter through the cuff.

In one aspect, the supportive backing can comprise a flexible strap that can be wrapped around the vein. Alternatively, the supportive backing can comprise at least one rigid panel having a hinge portion for positioning the rigid panel to follow the contour of the wall of the vein. In either configuration, the supportive backing is non-elastic such that the inflation of the chamber or chambers does not cause the supportive backing to stretch and instead forces the chamber or chambers to expand against the walls of the vein to compress the vein.

In one aspect, the chamber can comprise at least one predefined fold line defining a hinge portion for bending or folding the chamber to position the chamber around the vein. In this configuration, the chamber can be folded along the fold lines to define a plurality of faces, which contact the rounded wall of the vein to apply pressure to the vein to reduce the inner diameter of the vein. In another aspect, the chamber can further comprise a plurality of grooves that define a plurality of creases to define a continuous surface for contacting the wall of the vein. Similarly, each chamber can comprise at least one expansion groove positioned at the edges of the chamber or chambers. The expansion groove allow the chamber to expand when inflated to press against the vein and fold tightly against the supportive backing when the chamber is deflated to allow the vein to fully expand.

A method for treating ED, according to an aspect of the present invention, comprises positioning a cuff around at least one vein exiting from the corpus cavernosum, wherein the cuff comprises at least one inflatable chamber positioned against a wall of the vein. The method further comprises positioning a supportive cuff around the exterior of each chamber to prevent outward expansion of the chamber during inflation. The method also comprises inflating each chamber to apply pressure to the wall of the vein to decrease the inner diameter of the vein so as to create back pressure within the corpus cavernosum to inflate and maintain the corpus cavernosum at a predetermined pressure. The method also comprises deflating each chamber to remove the pressure on the wall of the vein to allow the inner diameter of the vein to return to the normal size and lower the pressure of the blood within the corpus cavernosum.

The above summary of the invention is not intended to describe each illustrated embodiment or every implementation of the present invention. The Figures and the Detailed Description that follow more particularly exemplify these embodiments.

### BRIEF DESCRIPTION OF THE DRAWINGS

The invention can be more completely understood in consideration of the following detailed description of various embodiments of the invention in connection with the accompanying drawings, in which:

FIG. 1 is a schematic view of a representative circulatory system of a male reproductive system for use with the present invention.

FIG. 2 is a schematic view of the representative circulatory system depicted in FIG. 1 with a constrictive cuff, according to an embodiment of the present invention, positioned around a vein of the circulatory system.

FIG. 2A is an enlarged partial cross-sectional view of the constrictive cuff depicted in FIG. 2.

FIG. 3 is a perspective view of a constrictive cuff according to an embodiment of the present invention in a rolled configuration.

FIG. 4 is a front view of a constrictive cuff according to an embodiment of the present invention in an unrolled configuration.

FIG. 5 is a front view of a constrictive cuff, according to an embodiment of the present invention, positioned around a representative vein.

FIG. 6 is a front view of the constrictive cuff depicted in FIG. 5 removed from the representative vein and in an unrolled configuration.

FIG. 7 is a front view of a constrictive cuff according to an embodiment of the present invention.

FIG. 8 is a front view of a constrictive cuff, according to an embodiment of the present invention, positioned around a representative vein.

FIG. 9 is a front view of the constrictive cuff depicted in FIG. 8 removed from the representative vein and in an unrolled configuration.

FIG. 10 is a top view of a constrictive cuff, according to an embodiment of the present invention.

FIG. 11 is a front view of the constrictive cuff depicted in FIG. 10 in an unrolled configuration.

FIG. 12 is a top view of a constrictive cuff, according to an embodiment of the present invention.

FIG. 13 is a front view of the constrictive cuff depicted in FIG. 10 in an unrolled configuration.

FIG. 14 is a front view of a constrictive cuff, according to an embodiment of the present invention in a rolled configuration.

FIG. 15 is a front view of the constrictive cuff depicted in FIG. 8 and in an unrolled configuration.

FIG. 16 is a side view of a constrictive cuff, according to an embodiment of the present invention in an unrolled configuration.

FIG. 17 is a cross-sectional side view of the constrictive cuff depicted in FIG. 16.

FIG. 18 is a schematic view of the representative circulatory system depicted in FIG. 1 with a valve device, according to an embodiment of the present invention, positioned around an artery and a vein of the circulatory system.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF PREFERRED EMBODIMENTS

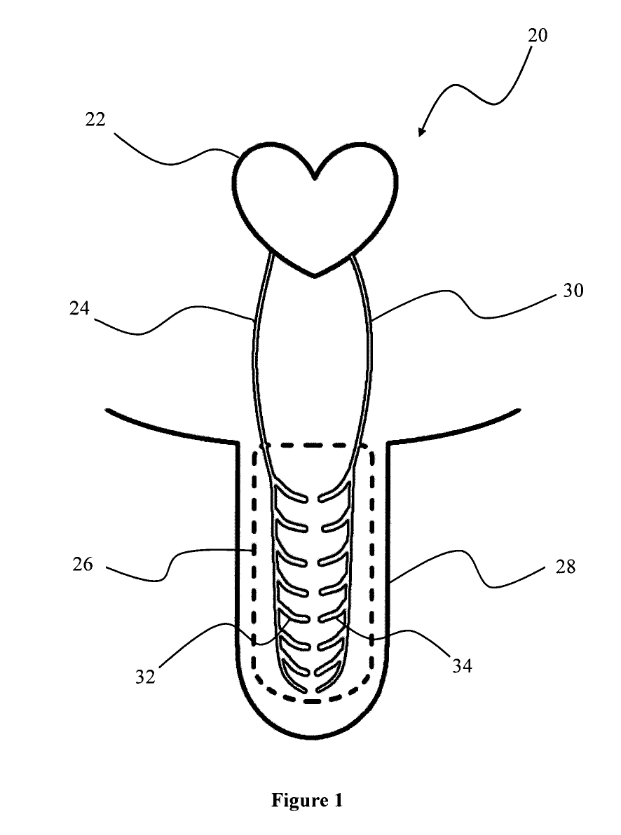

As shown in FIG. 1, a representative circulatory system 20 for a male reproductive system generally comprises a heart 22, an artery 24, two corpus cavernosum 26 within the penis 28 and a vein 30. Typically, the heart 22 operates to pump blood through the artery 24 into the corpus cavernosum 26 to inflate the corpus cavernosum 26 to create an erection. The artery 24 can further comprise a plurality of arterial branches 32 within the corpus cavernosum 26 for evenly distributing the blood. Similarly, the vein 30 can also comprise a plurality of vein branches 34 for collecting blood within the corpus cavernosum 26 to return the blood to the heart. During an erection, blood is still circulated into the corpus cavernosum 26 from the artery 24 and out of the corpus cavernosum 26 through vein 30. The description of the circulatory system 20 is not intended to be limiting, but is provided simply to assist in the description of the present invention.

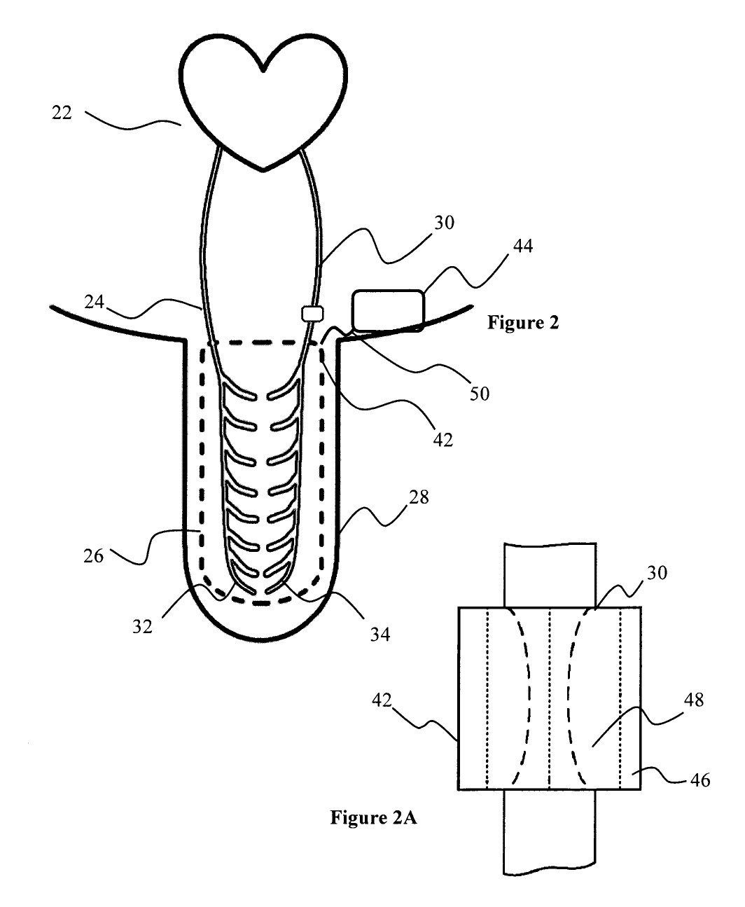

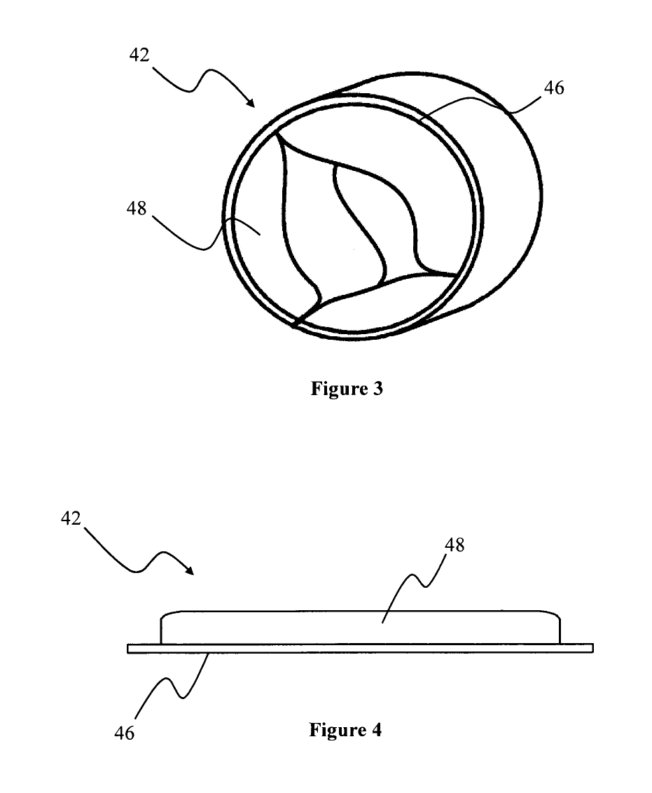

As shown in FIG. 2, an implantable ED treatment device 40, according to an embodiment of the present invention, comprises at least one constrictive cuff 42 and an inflation system 44. As shown in FIGS. 3 to 4, each constrictive cuff 42 further comprises a supportive backing 46 and at least one inflatable chamber 48. In one aspect, each cuff 42 can comprise multiple inflatable chambers 48 positioned along the length of the supportive backing 46 as shown in FIG. 3. Alternatively, each cuff 42 can comprise a single inflatable chamber 48 extending along the length of the supportive backing 46 as shown in FIG. 4. In one aspect, the inflation system 44 can be operably linked to the constrictive cuff 42 by kink resistant tubing 50 such that the inflation system 44 can inflate each chamber 48 with a working gas or fluid.

As shown in FIGS. 2, 2A and 3-4, the cuff 42 is foldable between a rolled configuration and an unrolled configuration. In the rolled configuration, the cuff 42 can be positioned around the vein 30 such that each inflatable chamber 48 is positioned against the vein 30. As shown in FIG. 2A, each inflatable chamber 48 can be inflated to compress against the vein 30 to reduce the inner diameter of the vein 30. The expansion of the inflatable chamber 48 limited to reduce the inner diameter of the vein 30 without fully closing off the vein 30 preventing over pressuring of the corpus cavernosum 26. In one aspect, the cuff 42 can comprise stops, sensors, and other limiting mechanisms or techniques can be employed to ensure that the blood flow is monitored and/or not completely stopped. The supportive backing 46 is non-elastic such that each inflatable chamber 48 only expands inward toward the vein 30. The reduced inner diameter of the vein 30 increases the pressure within the corpus cavernosum 26 to create an erection. Similarly, each inflatable chamber 48 can be deflated to allow the vein 30 to return to the normal diameter and allow the corpus cavernosum 26 to return to the normal pressure.

A method for treating ED according to an aspect of the present invention comprises positioning the cuff 42 having the at least one inflatable chamber 48 around the vein 30 such that the inflatable chamber 48 is positioned against the vein 30. The method further comprises inflating each inflatable cylinder 48 to constrict the vein 30 creating back pressure within the corpus cavernosum 26. Finally, the method comprises deflating each chamber 48 such that the vein 30 returns to its original diameter.

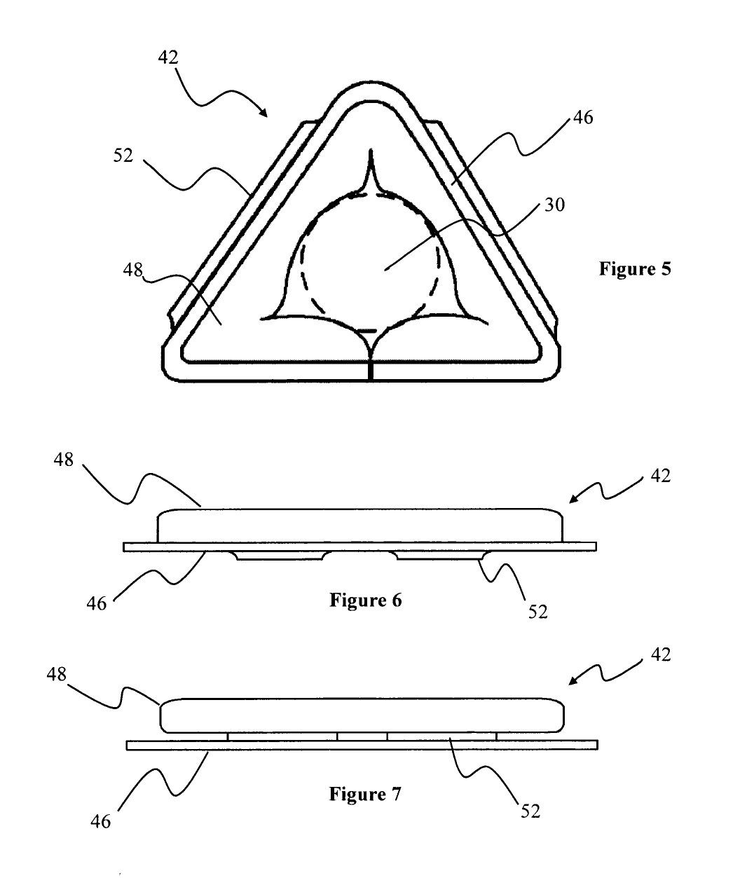

As shown in FIGS. 5 to 7, according to an aspect, the cuff 42 can further comprise at least one rigid panel 52 positioned on the supportive backing 46 for maintaining the shape of the cuff 42. In one aspect, the rigid panels 52 can be positioned on the exterior of the supportive backing 46 as shown in FIGS. 5 to 6. In another aspect, the rigid panels 52 can be positioned between the chamber 48 and the supportive backing 46 as shown in FIG. 7. In either configuration, the supportive backing 46 comprises a flexible material.

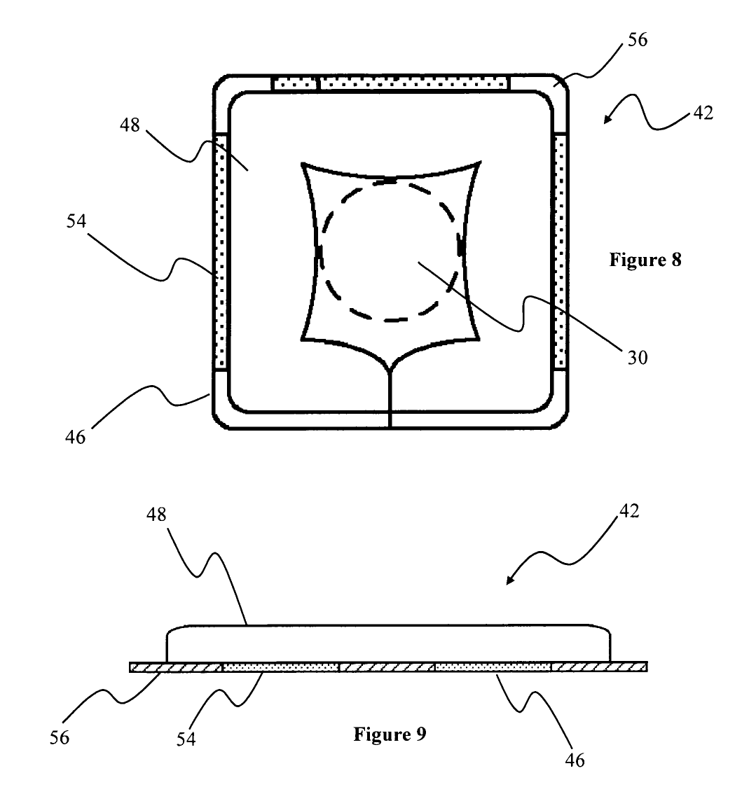

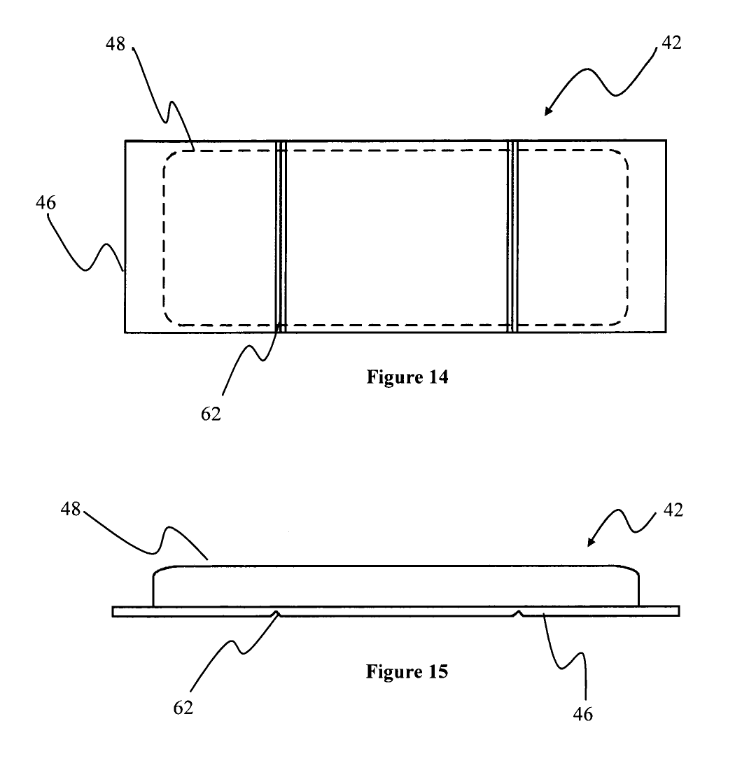

As shown in FIGS. 8 to 9, in one aspect, the supportive backing 46 can comprise a plurality of rigid sections 54 and flexible section 56. The rigid sections 54 and flexible sections 56 are alternated such that the flexible sections 56 can be bent such that the rigid sections 54 define the sides of a polygonal shape. The rigid sections 54 support the cuff 42 to maintain the shape of the cuff 42 when the cuff 42 is positioned around the vein 30. Similarly, as shown in FIGS. 14 to 15, in one aspect, the supportive backing 46 can comprise at least one fold line 62 defining a hinge portion along which the supportive backing 46 can be folded. In this configuration, the supportive backing 46 can comprise a rigid material to maintain the shape of the cuff 42 once wrapped around vein 30.

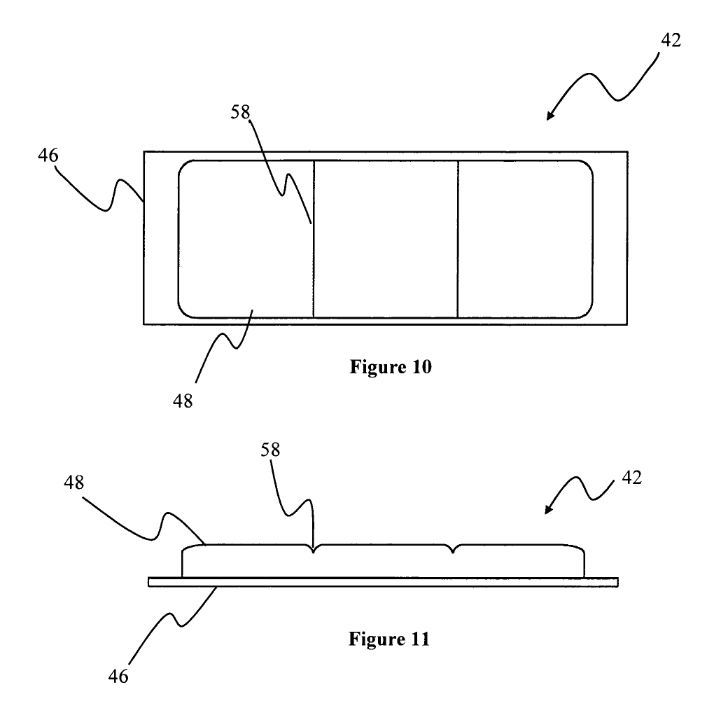

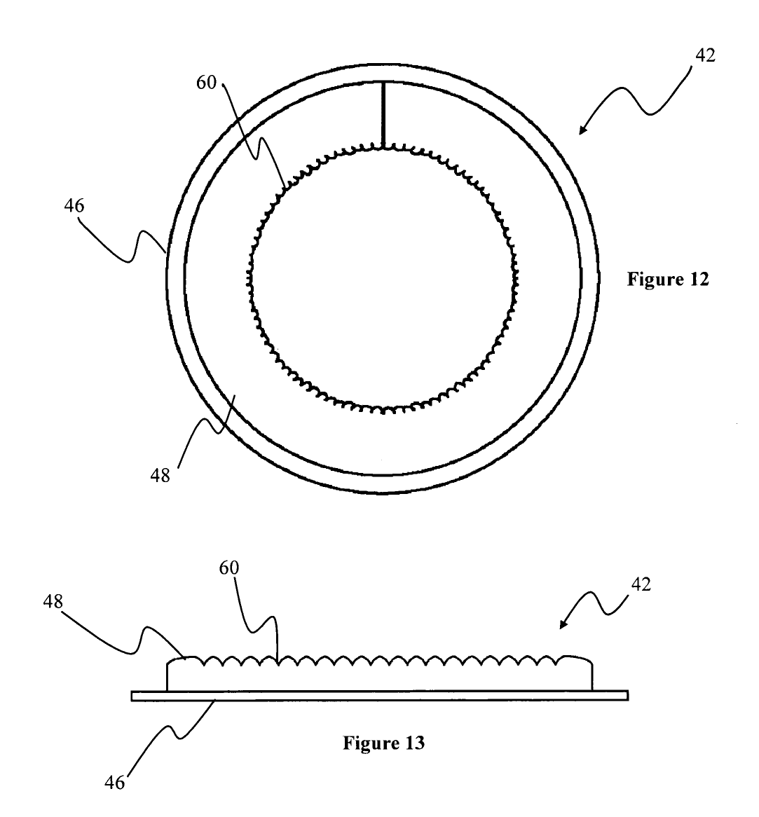

As shown in FIGS. 10 to 11, in one aspect, the inflatable cushion 48 can comprise at least one fold line 58 to form a crease in the inflatable cushion 48 when cuff 42 is positioned around a vein 30. The inflatable cushion 48 can often crease when the cuff 42 is wrapped around the vein 30. The creases can be formed too close together such that the cushion 48 does not evenly press against the vein 30 as the cushion 48 is inflated. The fold line 58 forms predefined creases such that the creases are evenly spaced around the vein 30. Similarly, as shown in FIGS. 12 to 13, in one aspect, the inflatable cushion 48 can comprise a plurality of grooves 60 defined along the entire surface of the cushion 48. The grooves 60 are positioned such that the inflatable cushion 48 provides continuous contact to the inflatable cushion 48 to prevent damage to the vein 30.

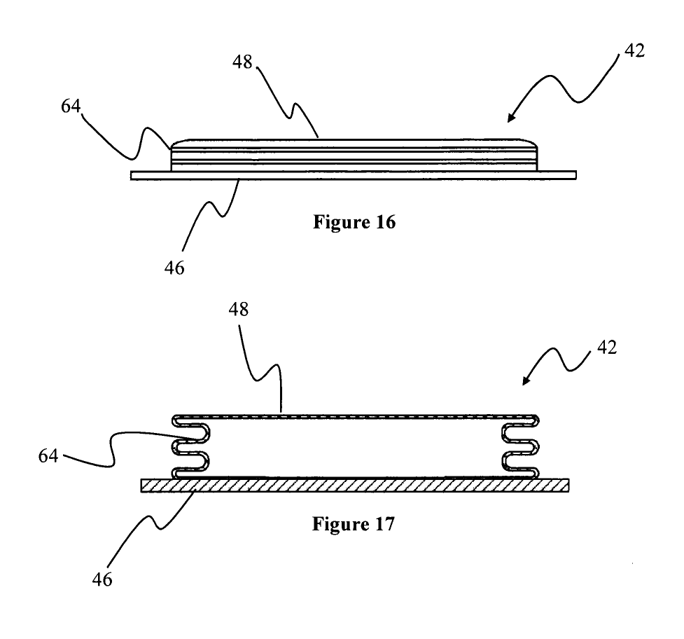

As shown in FIGS. 16 to 17, the inflatable cushion 48 can further comprise at least one expansion crease 64 defined at the edges of the inflatable cushion 48. The creases 64 are adapted to open when the cushion 48 is inflated to allow the cushion 48 to expand. Similarly, the creases 64 are adapted to reform when the cushion 48 is deflated to allow the vein 30 to fully re-expand.

In one aspect, the cuff 42 can further comprise actuation devices, automated sensors and like devices to control the restriction level and timing of the cuff 42. The relative constriction of the vein 30 controls the pressure within the corpus cavernosum 26. The constriction of the cuff 42 can be adjusted such that the relative pressure within the corpus cavernosum 26 can be increased or decreased depending on the patient's preference. Similarly, the constriction of the cuff 42 can be adjusted to adapt the operation of the cuff 42 for each patient's unique physiology allowing for consistent performance across multiple patients.

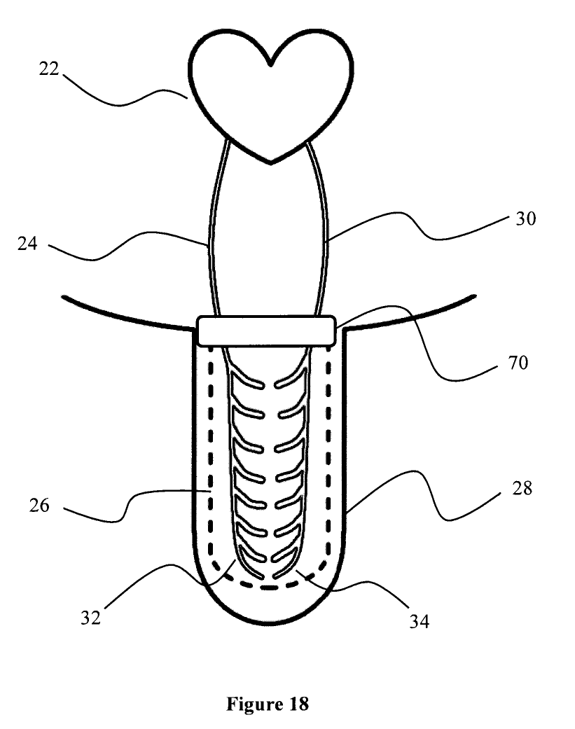

As shown in FIG. 18, in one aspect, the present invention can include a valve 70 or other regulation device in combination with or in lieu of the micro cuff 42. The valve device 70 can regulate and direct flow through the artery into the corpus cavernosum 26. The valve device 70 can also control the flow from the vein from the corpus cavernosum. During an erection, the valve device 70 can close off or limit blood flow out of the penis, as shown in FIG. 2. In a non-erect or flaccid condition, the valve device 70 can remains open to enable blood to freely flow out of the corpus cavernosum 26. In one aspect, the valve 70 can also constrict the artery 24 to control the flow of the blood into the corpus cavernosum 26 to maintain the cavernosum 26 at a predetermined pressure.

A variety of materials may be used to form portions or components of the present invention, including nitinol, polymers, elastomers, thermoplastic elastomers, metals, ceramics, springs, wires, plastic tubing, and the like.

All patents, patent applications, and publications cited herein are hereby incorporated by reference in their entirety as if individually incorporated, and include those references incorporated within the identified patents, patent applications and publications. Obviously, numerous modifications and variations of the present invention are possible in light of the teachings herein. It is therefore to be understood that within the scope of the appended claims, the invention may be practiced other than as specifically described herein.

1. A cuff for the treatment of erectile dysfunction, comprising:
a supportive backing;
at least inflatable chamber mounted along a length of the supportive backing such that the at least one inflatable chamber is inflatable to constrict a vein leading from a corpus cavernosum.

2. The cuff of claim 1, wherein the supportive backing comprises a non-elastic material such that the inflation of each chamber does not cause outward expansion of the cuff.

3. The cuff of claim 1, wherein the supportive backing further comprises at least one rigid panel for maintaining the shape of the cuff when positioned around the vein.

4. The cuff of claim 3, wherein the at least one rigid panel is positioned between the supportive backing and the at least one inflatable chamber.

5. The cuff of claim 1, wherein the supportive backing further comprises at least one rigid section for maintaining the shape of the cuff when positioned around the vein and at least one flexible section for defining a hinging portion for folding the cuff around the vein.

6. The cuff of claim 5, wherein the at least one rigid section and the at least one flexible section are arranged in an alternating arrangement.

7. The cuff of claim 1, wherein the supportive backing further comprises at least one fold line defining a hinging portion for folding the cuff around the vein.

8. The cuff of claim 1, wherein the at least one inflatable chamber defines at least one fold line for forming a crease when inflatable chamber is folded to position the cuff around the vein.

9. The cuff of claim 1, wherein the at least one inflatable chamber defines a plurality of grooves for forming a plurality of creases when inflatable chamber is folded to position the cuff around the vein, wherein the grooves are spaced such that the inflatable chamber contours to vein.

10. A system for treating erectile dysfunction comprising:
a cuff having a supportive backing and at least one inflatable chamber, wherein the cuff is positioned around a vein leading from a corpus cavernosum such that the at least one inflatable chamber is positioned against the vein; and
an inflation system for inflating the inflatable chamber;
wherein the at least one inflatable chamber is inflatable to constrict the vein to create a back pressure within the corpus cavernosum.

11. The system of claim 10, wherein the supportive backing comprises a non-elastic material such that the inflation of the at least one inflation chamber does not cause outward expansion of the cuff.

12. The system of claim 10, wherein the supportive backing further comprises at least one rigid panel for maintaining the shape of the cuff when positioned around the vein.

13. The system of claim 12, wherein the at least one rigid panel is positioned between the supportive backing and the at least one inflatable chamber.

14. The system of claim 10, wherein the supportive backing further comprises at least one rigid section for maintaining the shape of the cuff when positioned around the vein and at least one flexible section for defining a hinging portion for folding the cuff around the vein.

15. The system of claim 14, wherein the at least one rigid section and the at least one flexible section are arranged in an alternating arrangement.

16. The system of claim 10, wherein the supportive backing further comprises at least one fold line defining a hinging portion for folding the cuff around the vein.

17. The implantable device of claim 10, wherein the at least one inflatable chamber defines at least one fold line for forming a crease when inflatable chamber is folded to position the cuff around the vein.

18. The implantable device of claim 10, wherein the at least one inflatable chamber defines a plurality of grooves for forming a plurality of creases when inflatable chamber is folded to position the cuff around the vein, wherein the grooves are spaced such that the inflatable chamber contours to the vein.

19. A method for the treating erectile dysfunction, comprising:
positioning a cuff having at least one inflatable chamber around a vein leading from a corpus cavernosum;
inflating the at least one inflatable chamber to constrict the vein and create a back pressure within the corpus cavernosum to assist in an erection; and
deflating the at least one inflatable chamber to restore ordinary blood flow through the vein.

20. The method of claim 19, wherein inflating the at least one inflatable chamber, further comprises:
limiting the inflation of the inflatable chamber to prevent fully closing blood flow through the vein.
